(19) United States
(12) Patent Application Publication
(10) Pub. No.: US 2008/0167664 A1
(43) Pub. Date: Jul. 10, 2008

(54) Method and apparatus for verifying occlusion of fallopian tubes

(75) Inventors: F. Mark Payne, Palo Alto, CA (US); Robert Kotmel, Burlingame, CA (US)

Correspondence Address: CYTYC CORPORATION, 250 CAMPUS DRIVE, MARLBOROUGH, MA, 01752, US

(73) Assignee: Cytyc Corporation, Marlborough, MA (US)

(21) Appl. No.: 11/953,752

(22) Filed: Dec. 10, 2007

Related U.S. Application Data
(60) Provisional application No. 60/869,704, filed on Dec. 12, 2006.

(51) Int. Cl. A61F 6/20

(52) U.S. Cl. 606/135

(57) ABSTRACT

A device for verifying occlusion of the fallopian tube in a female subject includes an elongate gas delivery member having a lumen disposed therein, the elongate gas delivery member adapted for sealing engagement with the subject's uterus. The device includes a pressurized insufflation gas source coupled to the elongate gas delivery member, the insufflation gas source being in communication with the lumen of the elongate gas delivery member. The insufflation gas may includes for example, carbon dioxide. In one aspect, the device includes a pressure sensor or gauge to measure intra-uterine pressure to verify occlusion of the fallopian tube(s). In another aspect, the flow rate of insufflation gas into the uterus may be measured using a flow meter to verify occlusion of the fallopian tube(s).

### FIELD OF THE INVENTION

The field of the invention generally relates to methods and devices used to verify or detect occlusion of a body lumen. More specifically, the field of the Invention pertains to methods and devices for detecting or verifying fallopian tube occlusion.

### BACKGROUND OF THE INVENTION

Conventionally, bilateral tubal sterilization (BTS) has been used for sterilization in female patients. Typically, BTS is surgically accomplished by ligation of the fallopian tubes using one or more surgical approaches. More recently, various non-operative methods of achieving sterility have been developed as an alternative to conventional BTS procedures. For example, Conceptus, Inc. of San Carlos, Calif., has developed the ESSURE micro-insertion device which is deployed hysteroscopically. Also, Adiana, Inc. of Redwood City, Calif., has developed a hysteroscopically-placed device which uses low level radiofrequency energy to damage the fallopian tubes. A soft polymer matrix is left behind in the tube to facilitate closure. In both of these processes, sterilization is accomplished by occlusion of the intramural portion of the fallopian tubes.

These new, non-operative methods require some sort of post-procedure verification to ensure that the fallopian tube(s) have indeed been occluded. Typically, occlusion is verified after the sterilization procedure with the aid of hysterosalpinography (HSG). HSG is a radiographic technique in which a contrast media (e.g., oil or water soluble fluid containing a radiographically opaque compound of a material such as iodine) is injected slowly into the uterine cavity and fallopian tubes via a transcervicallly-placed cannula. Radiographic images are taken to delineate the inside of the uterus and fallopian tubes. Tubal occlusion is verified by the lack of contrast media past a specific location in the tube (or by lack of contrast media in certain anatomical spaces such as the pouch of Douglas). Unfortunately, HSG subjects the patient to ionizing radiation and the patient may potentially be sensitive to the contrast medium. Also, because HSG involves radiation, the procedure must be performed in a specialized suite or room suitable for radioactive procedures.

More recently, hysterosalpingo-contrast sonography (HyCoSy) has been developed for imaging the uterus and fallopian tubes. HyCoSy is an ultrasonic technique that is accomplished transvaginally after the uterus and fallopian tubes are filled with contrast media. Tubal occlusion (or lack thereof is determined by the absence of contrast media past a specific location in the fallopian tube or by the absence of contrast media in other anatomical spaces (e.g., the pouch of Douglas). While HyCoSy does obviate the risks of radiation exposure, the method employs somewhat complex and expensive equipment. There is a need for a less complex device and method that can be used to verify and/or detect occlusions within the fallopian tube. Preferably the device and method should be able to verify occlusion in the intramural portion of the patient's fallopian tubes.

### SUMMARY

In one embodiment of the invention, a device for verifying occlusion of the fallopian tube in a female subject includes an elongate gas delivery member having a lumen disposed therein, the elongate gas delivery member adapted for sealing engagement with the subject's uterus. The device includes a pressurized insufflation gas source coupled to the elongate gas delivery member, the insufflation gas source being in communication with the lumen of the elongate gas delivery member. The insufflation gas may include, for example, carbon dioxide. The device includes a pressure gauge interposed between the pressurized insufflation gas source and a distal end of the elongate gas delivery member for monitoring insufflation gas pressure of the subject's uterine cavity. In an alternative embodiment, a pressure sensor may be affixed or otherwise incorporated into the elongate gas delivery member to measure intrauterine pressure.

In another embodiment of the invention, a device for verifying occlusion of the fallopian tube in a female subject includes an elongate gas delivery member having a lumen disposed therein, the elongate gas delivery member adapted for sealing engagement with the subject's uterus. The device includes a pressurized insufflation gas source coupled to the elongate gas delivery member, the insufflation gas source being in communication with the lumen of the elongate gas delivery member. A flow meter is interposed between the pressurized insufflation gas source and a distal end of the elongate gas delivery member for monitoring the flow rate of the insufflation gas into the subject's uterine cavity.

In still another embodiment of the invention, the device may include both the pressure gauge and the flow meter as described above. One or both of the pressure gauge and flow meter may be used to detect leakage of the insufflation gas past the region of the fallopian tube containing the occlusive device. For example, the measured flow rate required to keep a substantially constant pressure within the uterine cavity may be used to detect the presence or absence of any leaks across the putative occlusion. Alternatively, the pressure gauge may be monitored after charging the uterine cavity with a pressurized charge of insufflation gas. The decay or drop on pressure may be used to detect any leaks across the occlusion formed within the fallopian tubes.

In still another embodiment of the invention, a method of verifying the occlusion of a fallopian tube of a female subject includes the steps of providing a source of pressurized insufflation gas, the gas source being coupled to a delivery member that can be inserted into the uterine cavity so as to form a seal between the delivery member and the uterus. Pressurized insufflation gas is then delivered from the source to the uterine cavity. The pressure of the insufflation gas contained within the uterus is measured over a period of time to detect the presence or absence of fallopian tube occlusion. For example, the pressure drop over a period of time may be used to determine whether the fallopian tube(s) are indeed occluded. The threshold or cutoff levels for leakage rates may be determined experimentally.

In yet another embodiment of the invention, a method of verifying the occlusion of a fallopian tube of a female subject includes the steps of providing a source of pressurized insufflation gas, the gas source being coupled to a delivery member that can be inserted into the uterine cavity so as to form a seal between the delivery member and the uterus. Pressurized insufflation gas is then delivered from the source to the uterine cavity. After the uterine cavity has initially been charged, a small flow of insufflation gas may be metered into the cavity to maintain a substantially constant pressure. The flow rate (or volume) of this metered gas may be monitored to detect the presence or absence of fallopian tube occlusion. The threshold or cutoff levels used to determine whether or not the fallopian tube(s) are indeed occluded may be determined experimentally.

### BRIEF DESCRIPTION OF THE DRAWINGS

The drawings illustrate the design and utility of various embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a schematic representation of a device for verifying occlusion of the fallopian tube in a female subject according to one embodiment.

FIG. 2 is a schematic representation of a device for verifying occlusion of the fallopian tube in a female subject according to another embodiment.

FIG. 3 is a partial cross-sectional view of the female reproductive system showing placement of a gas delivery member according to one embodiment of the invention.

FIG. 4 is a partial cross-sectional view of the female reproductive system showing placement of a gas delivery member according to another embodiment of the invention.

FIG. 5 is a partial cross-sectional view of the female reproductive system showing placement of a gas delivery member according to still another embodiment of the invention.

FIG. 6 is a flowchart of a method of verifying occlusion of a fallopian tube of a female subject according to one embodiment.

### DETAILED DESCRIPTION

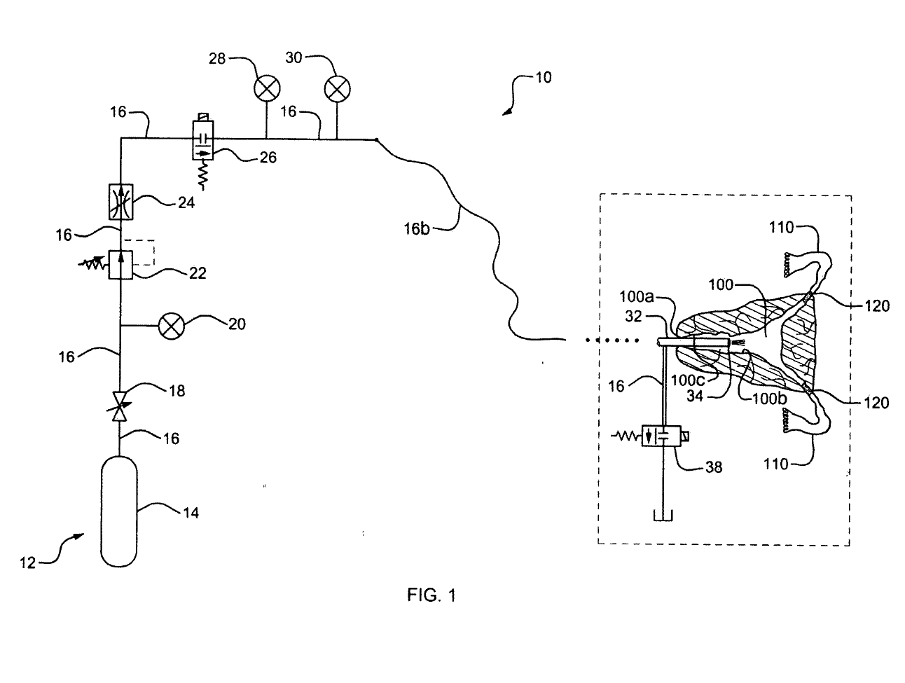

FIG. 1 illustrates an apparatus 10 for verifying whether or not a fallopian tube of a female subject is occluded. The apparatus 10 generally includes a source of pressurized insufflation gas 12. The insufflation gas 12 may include a gas such as, for example, USP grade carbon dioxide, although other gases may also be used in the apparatus 10. In the case of carbon dioxide, the insufflation gas 12 may be stored as a liquid and released in gaseous form. The pressurized insufflation gas 12 may be contained in a vessel or container 14 such as, for instance, a cylinder or tank commonly used in medical applications to store pressurized gases. In other embodiments, however, the apparatus 10 may be coupled to another source of pressurized gas. For example, hospitals and other medical facilities often have pressurized gas ports integrated into the construction of individual examination rooms.

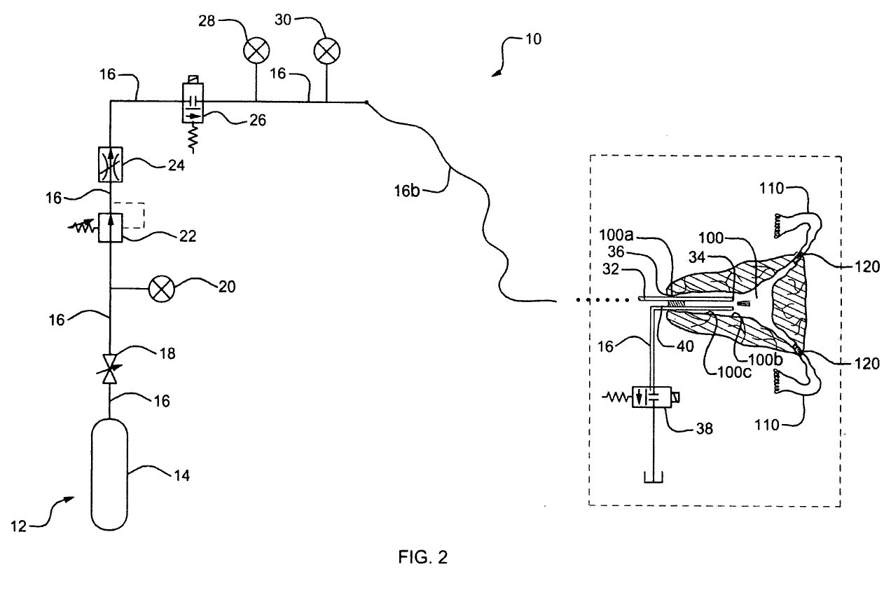

The apparatus 10 includes a conduit 16 that is used to connect or couple the various components of the apparatus 10. The conduit 16 includes an interior lumen through which the pressurized insufflation gas 12 can flow through. The conduit 16 may include tubing, piping, hose, or the like. The conduit 16 may be rather rigid or stiff in certain segments or regions while flexible in others. For example, conduit segment 16b in FIGS. 1 and 2 is made of a flexible hose or the like to permit manipulation of the gas delivery member (described in more detail below).

The tank 14 of pressurized insufflation gas 12 is coupled via the conduit 16 to a shut off valve 18. This shut off valve 18 can be used to stop all gas flow through the apparatus 10. The shut off valve 18 may be integrated with the tank 14 or it may be a separate component. The shut off valve 18 permits the removal and replacement of a tank 14 that may have a low reserve of insufflation gas 12. A downstream segment of conduit 16 connects the shut off valve 18 to a pressure gauge 20. The pressure gauge 20 is used to monitor the level or quantity of insufflation gas 12 remaining in the container 14. In addition, the pressure gauge 20 indicates to the operator when the main shut of valve 18 has been opened or closed. Downstream of the pressure gauge 20, another conduit segment 16 connects to a pressure regulator 22. The pressure regulator 22 is adjustable by the operator and permits the occlusion verification tests described herein to be performed at a multitude of pressures. In this regard, the particular pressure applied to the uterine cavity 100 (shown in FIGS. 1-5) can be adjusted by the operator. The pressure regulator 22 may include dial or indicator of the pressure so that the operator can quickly and accurately adjust the pressure of the apparatus 10.

Still referring to FIG. 1, a conduit 16 connects the downstream gas flow from the pressure regulator 22 to a flow control valve 24. The flow control valve 24 is used control the flow rate of the insufflation gas 12 into the uterine cavity 100. For example, FDA standards for hysteroscopic insufflation require flow rates of less than 100 ml/minute. The flow control valve 24 can thus be used to raise or lower the flow rate of the insufflation gas 12 as needed. Gas from the flow control valve 24 continues via conduit 16 to a valve 26 that modulates the flow through the apparatus 10. The valve 26 operates in either an “off” state or an “on” state. The valve 26 may include a powered solenoid valve that, when energized, permits insufflation gas 12 to flow into the uterine cavity 100. In contrast, when the solenoid valve is not energized, insufflation gas 12 cannot pass the valve 26. The state of the valve 26 may be controlled through electronic circuitry (not shown) that is coupled to switch, button, or the like that is used to trigger gas insufflation. Such circuitry is well known to those skilled in the art and is not described herein.

In certain embodiments of the invention, the valve 26 may be used to isolate the apparatus 10. For example, if pressure is being monitored within the uterine cavity 100 (or within the system as a proxy for uterine cavity pressure), the valve 26 may be switched to an “off” state after the uterine cavity 100 has been pressurized with insufflation gas 12. The decay or loss of pressure within the system can then be monitored to detect or verify occlusion of the subject's fallopian tubes 110.

Still referring to FIG. 1, a conduit 16 connects the downstream output of the valve 26 to a pressure gauge 28 and flow meter 30. The pressure gauge 28 is used to measure the pressure within the uterine cavity 100. The actual point of measurement, however, may be outside the uterine cavity 100 as is shown in FIGS. 1 and 2. Generally, it is not expected that there would be a large pressure drop from the location of the pressure gauge 28 in FIGS. 1 and 2 and the pressure contained within the uterine cavity 100. Consequently, the pressure taken proximally with respect to the outlet of the apparatus 10 is thought to be an accurate estimate of the actual pressure experienced within the uterine cavity 100. The pressure gauge 28 may be an analog pressure gauge or even one with a digital readout or output that could be displayed on monitor or computer. In other embodiments, however, the pressure gauge 28 may measure pressure directly within the uterine cavity 100 using a small semiconductor, piezoelectric, or Micro-Electro-Mechanical Systems (MEMS) based pressure sensor. In this regard, the pressure gauge 26 may be integrated into the gas delivery member 32 which is described in detail below).

In certain embodiments, only the pressure gauge 28 is needed to detect or verify occlusion of the fallopian tubes 110. For example, as explained above, the uterine cavity 100 may be charged with a pressurized volume of insufflation gas 12. The solenoid valve 16 can then be turned to the “off” state and the pressure gauge 28 can be monitored to detect any leaks. Any leaks within the fallopian tube(s) 110 are detected be a reduction in measured pressure. The reduced pressure is caused by insufflation gas 12 passing the region of the fallopian tube 110 containing the occlusive device 120 and exiting out of the fallopian tube 110 and into the peritoneum cavity. For example, the presence of a leak between the occlusive device 120 and the fallopian tube 100 may be determined if the pressure drops above a certain threshold rate (e.g., mmHg/sec). In certain embodiments, some leakage within the system may be attributed to leakage between the uterine cavity 100 and the gas delivery member (described below) if the seal is not complete. Consequently, there may be a background or baseline level of pressure decay within the system even if the occlusive device(s) 120 have completely occluded the fallopian tubes 110. In this case, the natural or background rate of leakage may be determined and leakage rates falling above this level may be used to verify the presence or absence of any leaks.

As an alternative to using the pressure gauge 28, the apparatus 10 may employ a flow meter 30 to verify or detect occlusion of the fallopian tubes 110. In this embodiment, the uterine cavity 100 is charged with pressurized insufflation gas 12 to a target or set point pressure. The system 10 then supplies additional insufflation gas 12 to the uterine cavity 100 to maintain the target pressure. The flow rate of the additional insufflation gas 12 needed to maintain a substantially constant pressure within the uterine cavity 110 can then be used to verify occlusion of the fallopian tubes 110. For example, the presence of a leak can be made once the rate of gas flow (or volume) exceeds a certain threshold value. For example, there may be some slight leakage between the gas delivery member (described below) and uterine cavity 100. Additional leakage beyond this baseline level can be detected by additional flow needed within the apparatus 10 to maintain the pressure within the uterine cavity 100.

In this embodiment, the pressure within the uterine cavity 100 may be determined using the pressure gauge 28 described above, or alternatively, a pressure gauge 28 contained on or in the gas delivery member that is used to measure the pressure directly within the uterine cavity 100. The flow control valve 24 may be arranged in a feedback loop with the pressure gauge 28 (or other pressure sensor) such that the flow of insufflation gas 12 can automatically adjusted based on real time or near real time measurements of pressure within uterine cavity 100.

As seen in FIG. 1, a flexible conduit 16b such as a hose or tubing connects the proximal aspects of the device 10 to a gas delivery member 32. The gas delivery member 32 may be an elongate tubular member having one or more lumens 34 contained therein that are used as a passageway for the insufflation gas 12. The gas delivery member 32 may be formed as a catheter or cannula that is sized for insertion into the uterine cavity 100. For example, the gas delivery member 32 may take the form of a Foley-type catheter. The catheter or cannula may be dimensioned to have an external diameter such that a substantially airtight seal is formed between the gas delivery member 32 and the uterine cavity 100. The gas delivery member 32 may form a seal the external os 100a of the uterus, the internal os 100b of the uterus, or the cervical canal 100c or a combination thereof. In one aspect, as seen in FIG. 2, the gas delivery member 32 may include a sealing member 36 that aids in forming the seal with the uterine cavity 100. The sealing member 36 may include a pliable or resilient member that is disposed about the periphery of the gas delivery member 32. In yet another alternative, the sealing member 36 may including an expandable member such as, for instance, an inflatable balloon or the like that is affixed to the gas delivery member 32.

Still referring to FIG. 1, the lumen 34 of the gas delivery member 32 is coupled to a conduit 16 that communicates with a purge valve 38. Activation of the purge valve 38 enables the evacuation of insufflation gas 12 from the uterine cavity 100. The purge valve 38 may take the form of a solenoid valve that is activated electronically. Preferably, the conduit 16 connecting to the lumen 34 of the gas delivery member 32 to the purge valve 38 is located on the gas delivery member 32 at allocation that lies outside the patient. The connecting conduit 16 may even connect somewhere further on the proximal end of the gas delivery system.

FIG. 2 illustrates an alternative embodiment of the apparatus 10 in which the gas delivery member 32 is separate from an evacuation member 40. In FIG. 2, both the gas delivery member 32 and the evacuation member 40 pass through a common sealing member 36 although separate sealing members 36 could be used for each member 32, 40. The embodiment in FIG. 2 is different from that disclosed in FIG. 1 in there is no common lumen that both delivers and evacuates insufflation gas 12 into and out of the uterine cavity 100.

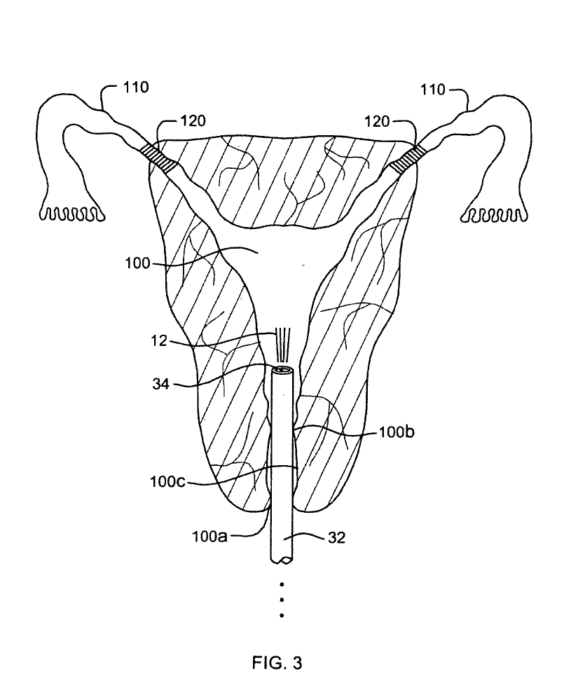

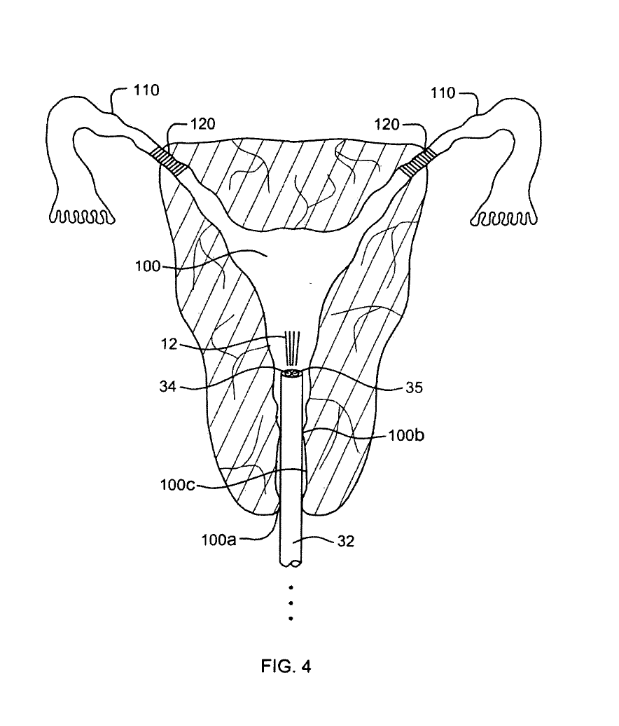

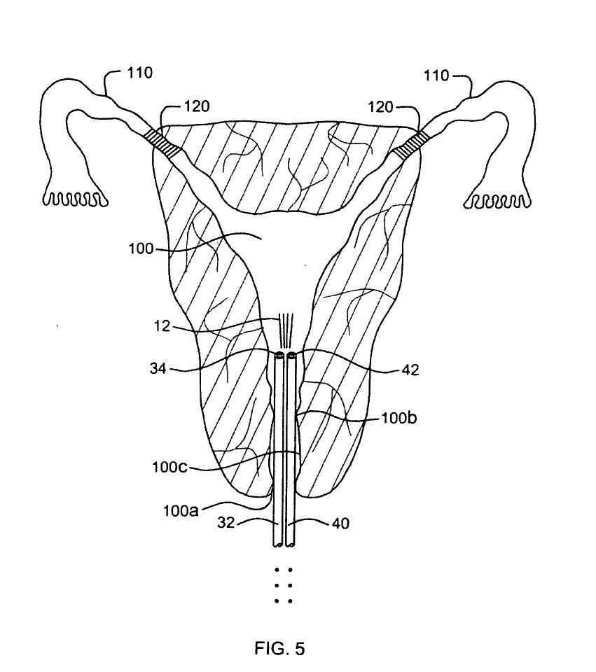

It should be understood that a variety of designs may be employed for the gas delivery member 32. For example, FIG. 3 illustrates a view of the deployed gas delivery member 32 inside the uterine cavity 100. The gas delivery member 32 includes a single lumen 34 that is used for both delivery and evacuation of insufflation gas 12. FIG. 4 illustrates a dual lumen embodiment of a gas delivery member 32 which has a first lumen 34 for insufflation gas delivery and a second lumen 35 for insufflation gas evacuation. FIG. 5 illustrates yet another embodiment that uses a separate evacuation member 40. The evacuation member 40 includes its own lumen 42 for gas evacuation.

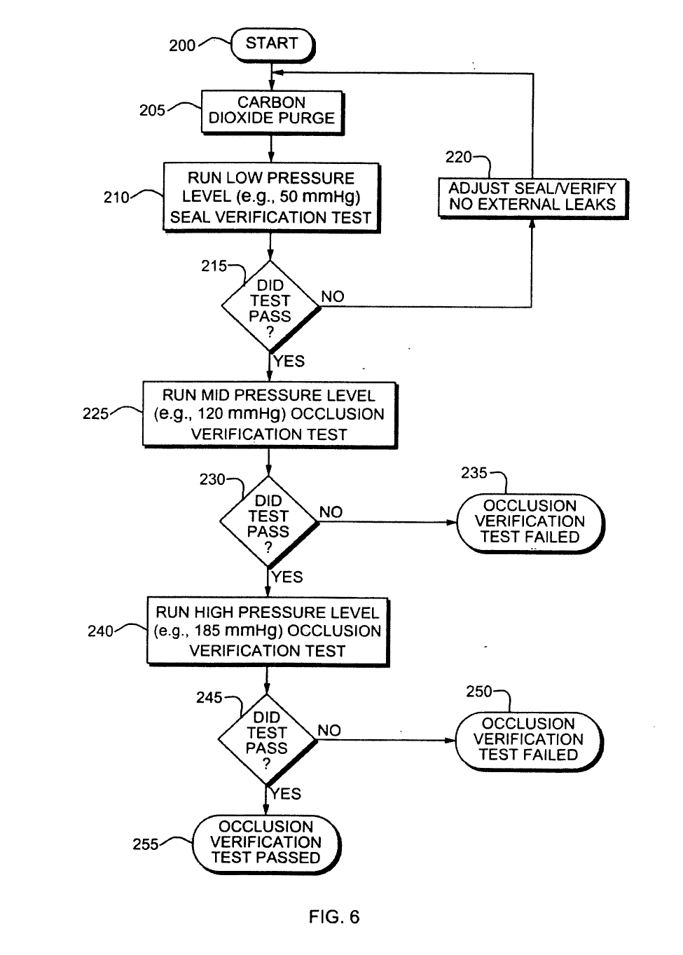

FIG. 6 illustrates an exemplary flow diagram showing one embodiment of the operation of the device 10. Initially, as seen in step 200, the device 10 is started by connecting the various components and ensuring that the same are operational. Next, in step 205 the device 10 undergoes a purge process to flush the system with insufflation gas 12 (e.g., carbon dioxide). The gas delivery member 32 is then inserted into the uterine cavity 100 transvaginally by the operator. Alternatively, the purge process may be initiated after insertion of the device 10 into the patient. In yet another alternative, the purge process may take both before and after placement of the device 10. During the placement process, the subject may be placed into the lithotomy position with knees raised and the cervix exposed using a standard speculum or the like. The gas delivery member 32 can then be advanced within the subject's cervix.

As seen in step 210, a low pressure test is then run to determine whether or not a proper seal has been formed between the gas delivery member 32 and the uterus. For example, a low pressure of about 50 mmHg insufflation gas 12 may be delivered to check for system leaks. Assuming a leak was detected, as illustrated in the pass query step 215, the operator then adjusts the seal and/or placement of the gas delivery member 32 and checks for other sources of leaks within the system (step 220). The low pressure seal test (step 210) is then performed again. After the device 10 passed the low pressure test, a mid-level pressure is then delivered to the uterine cavity 100 to verify occlusion of the fallopian tubes 110 as is shown in step 225 of FIG. 6. The mid-level pressure may include an applied pressure of around 120 mmHg. Occlusion of the fallopian tubes 110 may be verified or confirmed using either the pressure or flow methods discussed herein.

Next, as seen in step 230 of FIG. 6, a query is made whether or not the test was passed. In this regard, if a leak was detected, the user would be notified that complete occlusion of the fallopian tubes 110 was not verified and the verification step failed (step 235). Assuming that the mid-level pressure test was successfully passed—thereby indicating that the fallopian tubes were fully occluded when subject to the mid-level pressure, the subject is then tested at a higher pressure level as is shown in step 240 in FIG. 6. The higher pressure level may include a pressure on the order of around 185 mmHg. It should be understood that the exact pressures described above with respect to the seal test and the mid and high pressure tests for fallopian tube occlusion may vary and still fall within the scope of the invention. Referring back to FIG. 6, another query is performed (step 245) to assess whether leaks were detected at the higher applied pressure. If leaks were detected, then the operator would be notified that the verification test failed (step 250). However, if no leaks were detected at the higher applied pressure, then the subject is said to have passed the occlusion verification test (step 255). In step 255, the patient is assured that the fallopian tubes 110 have indeed been fully occluded.

The device 10 described herein has been described in the context of testing both fallopian tubes 110 at the same time for determining whether total occlusion has occurred. In another embodiment of the invention, it may be possible to isolate one of the two fallopian tubes 110 for testing. For example, an inflatable member such as an inflatable balloon or the like may be used to seal off one of the fallopian tubes 100 such that the other fallopian tube 110 can be tested at a single time.

While embodiments of the present invention have been shown and described, various modifications may be made without departing from the scope of the present invention. The invention, therefore, should not be limited, except to the following claims, and their equivalents.

What is claimed is:

1. A device for verifying occlusion of the fallopian tube in a female subject comprising:
an elongate gas delivery member having a lumen disposed therein, the elongate gas delivery member adapted for sealing engagement with the subject's uterus;
a pressurized insulation gas source coupled to the elongate gas delivery member, the insufflation gas source being in communication with the lumen of the elongate gas delivery member; and
a pressure gauge interposed between the pressurized insufflation gas source and a distal end of the elongate gas delivery member for monitoring insufflation gas pressure of the subject's uterine cavity.

2. The device of claim 1, wherein the elongate gas delivery member comprises a catheter.

3. The device of claim 1, wherein the elongate gas delivery member comprises a cannula.

4. The device of claim 1, wherein the elongate gas delivery member sealingly engages with the subject's internal os.

5. The device of claim 1, wherein the elongate gas delivery member sealingly engages with the subject's cervical canal.

6. The device of claim 1, wherein the elongate gas delivery member sealingly engages with the subject's external os.

7. The device of claim 1, wherein the elongate gas delivery member sealingly engages with at least two of the subject's internal os, cervical canal, and external os.

8. The device of claim 1, further comprising a flow control valve disposed downstream of the pressurized insufflation gas source.

9. The device of claim 8, further comprising a solenoid valve disposed downstream of the flow control valve.

10. The device of claim 1, further comprising a purge valve coupled to the lumen of the elongate gas delivery member for evacuating gas from the uterine cavity of the patient.

11. The device of claim 1, the elongate gas delivery member including a second lumen for evacuating gas from the uterine cavity, the second lumen being operatively connected to a purge valve.

12. The device of claim 1, further comprising a monitor for reading pressure from the pressure gauge.

13. A device for verifying occlusion of the fallopian tube in a female subject comprising:
an elongate gas delivery member having a lumen disposed therein, the elongate gas delivery member adapted for sealing engagement with the subject's uterus;
a pressurized insufflation gas source coupled to the elongate gas delivery member, the insufflation gas source being in communication with the lumen of the elongate gas delivery member; and
a flow meter interposed between the pressurized insufflation gas source and a distal end of the elongate gas delivery member for monitoring the flow rate of insufflation gas into the subject's uterine cavity.

14. The device of claim 13, wherein the elongate gas delivery member comprises a catheter.

15. The device of claim 13, wherein the elongate gas delivery member comprises a cannula.

16. The device of claim 13, wherein the elongate gas delivery member sealingly engages with the subject's internal os.

17. The device of claim 13, wherein the elongate gas delivery member sealingly engages with the subject's cervical canal.

18. The device of claim 13, wherein the elongate gas delivery member sealingly engages with the subject's external os.

19. The device of claim 13, wherein the elongate gas delivery member sealingly engages with at least two of the subject's internal os, cervical canal, and external os.

20. The device of claim 13, further comprising a flow control valve disposed downstream of the pressurized insufflation gas source.

21. The device of claim 20, further comprising a solenoid valve disposed downstream of the flow control valve.

22. The device of claim 13, further comprising a purge valve coupled to the lumen of the elongate gas delivery member for evacuating gas from the uterine cavity of the patient.

23. The device of claim 13, the elongate gas delivery member including a second lumen for evacuating gas from the uterine cavity, the second lumen being operatively connected to a purge valve.

24. The device of claim 13, further comprising a monitor for reading pressure from the pressure gauge.

25. A device for verifying occlusion of the fallopian tube in a female subject comprising:
an elongate gas delivery member having a lumen disposed therein, the elongate gas delivery member adapted for sealing engagement with the subject's uterus;
a pressurized insufflation gas source coupled to the elongate gas delivery member, the insulation gas source being in communication with the lumen of the elongate gas delivery member;
a flow meter interposed between the pressurized insufflation gas source and a distal end of the elongate gas delivery member for monitoring the flow rate of insufflation gas into the subject's uterine cavity; and
a pressure gauge interposed between the pressurized insufflation gas source and a distal end of the elongate gas delivery member for monitoring insufflation gas pressure of the subject's uterine cavity.

26. The device of claim 25, wherein the elongate gas delivery member comprises a catheter.

27. The device of claim 25, wherein the elongate gas delivery member comprises a cannula.

28. The device of claim 25, wherein the elongate gas delivery member sealingly engages with the subject's internal os.

29. The device of claim 25, wherein the elongate gas delivery member sealingly engages with the subject's cervical canal.

30. The device of claim 25, wherein the elongate gas delivery member sealingly engages with the subject's external os.

31. The device of claim 25, wherein the elongate gas delivery member sealingly engages with at least two of the subject's internal os, cervical canal, and external os.

32. The device of claim 25, further comprising a flow control valve disposed downstream of the pressurized insufflation gas source.

33. The device of claim 32, further comprising a solenoid valve disposed downstream of the flow control valve.

34. The device of claim 25, further comprising a purge valve coupled to the lumen of the elongate gas delivery member for evacuating gas from the uterine cavity of the patient.

35. The device of claim 25, the elongate gas delivery member including a second lumen for evacuating gas from the uterine cavity, the second lumen being operatively connected to a purge valve.

36. The device of claim 25, further comprising a monitor for reading pressure from the pressure gauge.

37. A method of verifying occlusion of a fallopian tube of a female subject comprising:
providing a source of pressurized insufflation gas, the source of gas being coupled to a delivery member for sealingly engaging with the uterine cavity of the subject;
delivering pressurized insufflation gas from the source to the uterine cavity; and
measuring the pressure of the insulation gas contained within the uterine cavity over a period of time to detect the presence or absence of fallopian tube occlusion.

38. The method of claim 37, wherein the presence of fallopian tube occlusion is verified when the pressure is maintained above a threshold value for a period of time after pressurizing the uterine cavity.

39. The method of claim 37, wherein the presence of fallopian tube occlusion is verified when the rate of pressure drop exceeds a threshold value.

40. The method of claim 37, further comprising the step of purging the insufflation gas from the uterine cavity.

41. A method of verifying occlusion of a fallopian tube of a female subject comprising:
providing a source of pressurized insufflation gas, the source of gas being coupled to a delivery member for sealingly engaging with the uterine cavity of the subject;
delivering pressurized insufflation gas from the source to the uterine cavity at a target pressure; and
measuring the flow rate of the insufflation gas into the uterine cavity required to substantially maintain the target pressure in order to detect the presence or absence of fallopian tube occlusion.

42. The method of claim 41, wherein fallopian tube occlusion is verified when the flow rate of the insufflation gas into the uterine cavity is below a threshold value.

43. The method of claim 41, further comprising the step of purging the insufflation gas from the uterine cavity.
